(19) United States
(12) Patent Application Publication
(10) Pub. No.: US 2017/0270667 A1
(43) Pub. Date: Sep. 21, 2017

(54) ESTIMATING VECTORS OF SKIN PARAMETERS FROM A VIDEO OF EXPOSED SKIN

(71) Applicant: Xerox Corporation, Norwalk, CT (US)

(72) Inventors: Yasaman KHAZAENI, Brighton, MA (US); Lalit Keshav MESTHA, Fairport, NY (US); Alvaro Enrique GIL, Rochester, NY (US); Palghat Srinivas RAMESH, Pittsford, NY (US)

(21) Appl. No.: 15/070,379

(22) Filed: Mar. 15, 2016

(51) Int. Cl. G06T 7/00; A61B 5/1455; A61B 5/00; A61B 5/103; G06T 7/40; G06F 19/00

(52) U.S. Cl. CPC G06T 7/0014; G06T 7/408; G06F 19/322; G06F 19/321; A61B 5/443; A61B 5/0077; A61B 5/1032; A61B 5/7275; A61B 5/14551; A61B 5/7435; A61B 5/7475; G06T 2207/30088; G06T 2207/10024; G06T 2207/30096; G06T 2207/10016; G06T 2207/30024

(57) ABSTRACT

What is disclosed is a system and method for estimating a vector of skin parameters from in-vivo color measurements obtained from a video. In one embodiment, a video of exposed skin is received which comprises a plurality of time-sequential image frames acquired over time t. A vector of in-vivo color measurements is obtained on a per-frame basis from at least one imaging channel of a video imaging device used to capture the video. In a manner more fully disclosed herein, an intermediate vector of estimated skin parameters is determined based on an initial vector of estimated skin parameters and the in-vivo color measurements of all image frames averaged over time t. A final vector of estimated skin parameters is then determined for each image frame of the video based on the intermediate vector. The temporally successive final vectors are used to predict changes in time-varying skin parameters for the subject.

### TECHNICAL FIELD

The present invention is directed to systems and methods for estimating a plurality of vectors of skin parameters from in-vivo color measurements obtained from a video of an exposed skin surface which can thereafter be used to model various layers of skin.

### BACKGROUND

Skin cancers account for about 40% of all diagnosed cancers in humans. Most skin cancers are curable, if detected early enough. Suspicious lesions are biopsied for analysis. Biopsy can be an unpleasant experience for most patients. It is highly desirable in this art to be able to obtain accurate skin parameters which can be used to model various skin layers. The teachings hereof are specifically directed to this effort.

### BRIEF SUMMARY

What is disclosed is a system and method for estimating a plurality of vectors of skin parameters from in-vivo color measurements obtained from a video of an exposed skin surface. One embodiment hereof involves receiving a video of an area of exposed skin for processing. The image frames of the video comprise in-vivo color measurements of the skin obtained on a per-frame basis. In a manner more fully disclosed herein, an intermediate vector of estimated skin parameters is determined based on an initial vector of estimated skin parameters and the in-vivo color measurements of all image frames averaged over time t. Thereafter, a final vector of estimated skin parameters is determined for each frame of the video based on the intermediate vector of estimated skin parameters and the in-vivo color measurements of each respective image frame. The final vectors of estimated skin parameters are communicated to a storage device. The temporally successive final vectors are thereafter used to predict changes in time-varying skin parameters for the subject.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows one image frame of an unobstructed view of an area of exposed skin of a human hand being acquired by a video imaging device;

FIG. 2 is a functional block diagram of one example system for determining an intermediate vector of estimated skin parameters from a video of a skin surface;

FIG. 3 is a functional block diagram of one example system for determining a final vector of estimated skin parameters for each image frame of the video;

FIG. 4 is a flow diagram which illustrates one example embodiment of the present method for estimating a final vector of skin parameters for each image from of a video of a skin surface of a subject;

FIG. 5 is a continuation of the flow diagram of FIG. 4 with processing continuing with respect to nodes A or B;

FIG. 6 is a continuation of the flow diagram of FIG. 5 with processing continuing with respect to node D;

FIG. 7 is a continuation of the flow diagram of FIG. 6 with processing continuing with respect to node E;

FIG. 8 is a continuation of the flow diagram of FIG. 7 with processing continuing with respect to nodes F or G; and

FIG. 9 is a functional block diagram of one embodiment of an example video processing system for performing various aspects of the present method as described with respect to the block diagrams of FIGS. 2 and 3 and the flow diagrams of FIGS. 4-8.

### DETAILED DESCRIPTION

What is disclosed is a system and method for estimating a plurality of vectors of skin parameters from in-vivo color measurements obtained from a video of an exposed skin surface.

### Non-Limiting Definitions

A “subject” refers to a living being. Although the term “person” or “patient” may be used throughout this disclosure, the subject may be something other than a human such as, for example, a primate. Therefore, the use of such terms is not to be viewed as limiting the scope of the appended claims strictly to humans.

“Skin” protects underlying tissues, internal organs, and other anatomical structures against impact, abrasion, ultraviolet radiation, chemical exposure, to name a few. Skin accounts for approximately 16% of total body weight and comprises an epidermis, dermis, and hypodermis layer. The dermis layer lies between the epidermis and hypodermis layers and consists of multiple layers of cells with blood vessels, lymphatic structures, nerves, hair follicles, sweat glands, etc. The hypodermis layer is dominated by adipose tissue (subcutaneous fat) and serves as an insulating boundary. The epidermis is bloodless and dominated by epithelial cells that rely on diffusion of nutrients and oxygen from capillaries within the dermis layer. Melanocytes in the epidermis produce various shades of pigment called melanin which give color to the skin and which, in varying degrees, provides protection from ultraviolet radiation.

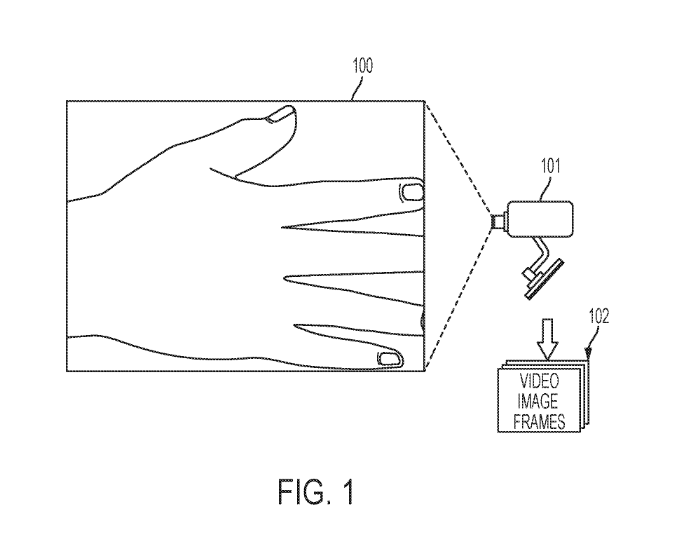

A “video” refers to a plurality of time-sequential image frames acquired over time t. Each image frame is a grid of pixels with each pixel having an associated color or intensity (color measurements) as detected by imaging elements of the video imaging device used to capture the video. Pixels in each of the image frames may be grouped, averaged, or processed as desired to obtain the in-vivo color measurements. The image frames may be pre-processed as needed to compensate for motion induced blurring, imaging blur, noise and other anomalies. The video may also contain other components such as, for instance, audio, time, date, reference signals, frame information, and the like. FIG. 1 shows an image frame 100 of an unobstructed view of an area of exposed skin of a human hand. The video is received for processing.

“Receiving a video” is intended to be widely construed and includes retrieving, capturing, acquiring, or otherwise obtaining video image frames for processing using the methods hereof. The video can be received from a remote device over a network, or from a media such as a CDROM or DVD. The video may be downloaded from a web-based system or application which makes video available for processing. Video can also be received from an application such as those which are available for handheld cellular devices and processed on the cellphone or other handheld computing device such as an iPad or Tablet-PC. The video can be received directly from the video imaging device used to capture that video.

A “video imaging device” is a color video camera, as is generally understood, which captures time-sequential image frames over a time duration t. The video imaging device can be any of a variety of devices available in various streams of commerce which may incorporate memory, a storage device, and one or more processors utilizing different hardware platforms. FIG. 1 shows an example RGB video camera 101 capturing image frames (collectively at 102) of an area of exposed skin of a right hand.

An “area of exposed skin” means an unobstructed view of the skin surface as seen through the aperture of the video camera used to capture video of that skin. An area of exposed skin may be automatically identified in a video using, for example, object identification, spatial features, color, shape, pattern recognition, pixel classification, and facial recognition methods. An area of exposed skin can be manually identified in a video by a technician using, for example, a mouse or a touchscreen to make a selection thereof from the video.

A “color space” encompasses all colors visible to the human eye.

A “device-Independent color space” refers to any standard color space used to describe color. Standard color spaces include CIE-XYZ, CIE-LAB, CIE-LUV, to name a few. CIE stands for Commission Internationale d'Eclairage (International Commission on Illumination).

A “device-dependent color space” is any non-standard color space that cannot be used to commonly define colors without additional information about the characteristics of the rendering device. The RGB space is a device-dependent color space since the rendering of an RGB image could appear differently from one device model to another. CMYK (Cyan, Magenta, Yellow, K=Black) is a device-dependent color space since the rendering of a given CMYK color could yield different (device-independent) colors from one printer to another printer. Methods for converting from one color space to another are well established in the color sciences.

An “initial vector of skin parameters Pᵢₙᵢₜ”, in one embodiment, is given by:

P=[fₑₚᵢ,fₘₑₗ,f_{blood},SO₂,Cₛ]^{T}

where fₑₚᵢ represents skin properties relating to epidermis thickness, fₘₑₗ represents skin properties relating to melanin concentration, f_{blood} represents skin properties relating to fractional of blood volume in the dermis layer, SO₂ represents skin properties relating to skin oxygen saturation, Cₛ represents the light scattering properties of skin tissue, and T is a transpose operation. In one embodiment, the initial vector of skin parameters contains at least one skin parameter which varies over the time duration t of the video and has at least one skin parameter which does not vary over time t. Time-varying skin parameters include dermal blood volume fraction, oxygen saturation, and a light scattering parameter. Time invariant skin parameters include epidermal thickness and melanin concentration.

“Generating a vector of skin parameters” can be effectuated using a variety of algorithms which include: Simultaneous Perturbation Stochastic Approximation (SPSA), Levenberg-Marquard Algorithm (LMA), Nelder-Mead Algorithm (NMA), Sequential Quadratic Programming (SQP), or a Genetic Algorithm, as are widely understood. Briefly, the SPSA is a descent method for finding global minima. Its main feature is the gradient approximation that requires only two measurements of an objective function, regardless of the dimension of the underlying optimization problem. It is widely used for optimizing systems with multiple unknown parameters. LMA provides a numerical solution to the problem of minimizing a function, generally nonlinear, over a space of parameters of the function. LMA interpolates between the Gauss-Newton Algorithm (GNA) and the method of gradient descent. LMA is typically more robust than GNA which means that, in many cases, it can find a solution even when it starts far off the final minimum. LMA is a popular algorithm used in many software applications for solving generic curve-fitting problems. However, LMA finds only a local minimum, not a global minimum. NMA is a commonly applied numerical method used to find the minimum or maximum of an objective function in a multidimensional space. It is applied to nonlinear optimization problems for which derivatives may not be known. This method approximates a local optimum of a problem with n variables when the objective function varies smoothly and is unimodal. SQP solves a sequence of optimization sub-problems each of which optimizes a quadratic model of the objective subject to a linearization of the constraints. If the problem is unconstrained then the method reduces to Newton's method for finding a point where the gradient of the objective vanishes. If the problem has only equality constraints, then the method is equivalent to applying Newton's method to the first-order optimality conditions. GA is a search heuristic that mimics the process of natural evolution. GA belongs to a larger class of Evolutionary Algorithms (EA) used to generate solutions to optimization and search problems. The generated vector of skin parameters is provided to a biophysics model.

A “biophysics model” is a semi-empirical Kubelka-Munk (K-M) model for diffuse reflectance from a surface of a finite layer which maps an input vector of skin parameters to the optical properties of skin and which generates, as output, a vector of estimated color measurements Cₑ in the form of reflectance spectra. The mapping is non-linear but may be approximated in a linear form given by:

$${C}_{e} = {\sum }_{i = 1}^{N} {P}_{i} {ϕ}_{i}$$

where Pᵢ is the i^{th} skin parameter of vector P, φᵢ is the i^{th} column-wise basis vector, and N is the number of skin parameters. The basis set is constructed by Design of Experiments (DOE) on the biophysics model or, for example, by Monte Carlo Simulation.

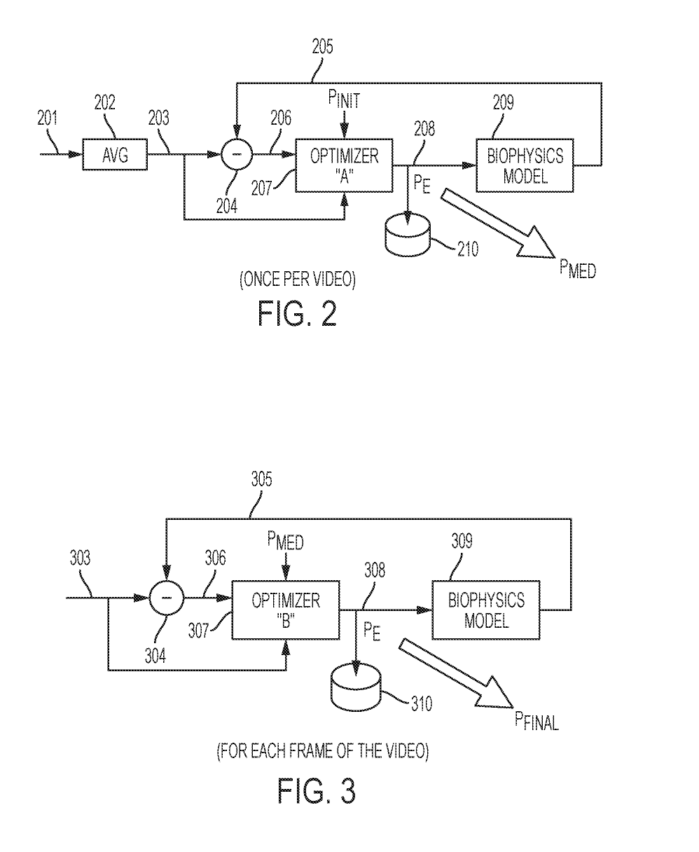

### Functional Block Diagram of FIG. 2

Reference is now being made to the functional block diagram of FIG. 2 which implements a system for determining an intermediate vector of estimated skin parameters. The initial vector of estimated skin parameters Pᵢₙᵢₜ is used on a first iteration to obtain a first vector of estimated color measurements 205.

In FIG. 2, in-vivo color measurements 201 obtained from all the image frames of the video of the skin surface are averaged 202 over time t to obtain a vector of time-averaged in-vivo color measurements 203. The vector of time-averaged in-vivo color measurements 203 is compared 204 against the vector of estimated color measurements 205 to determine if convergence has occurred. Convergence occurs when the error 206 between vector 203 and vector 205 is at or near zero. Convergence can be determined using, for example, a norm of an error vector, a sum of quadratic errors, a natural log of a sum of quadratic errors, a maximum square error, a mean square error, and a Kullback-Leibler divergence metric. If the color spaces of the two vectors being compared are different then the estimated color measurements can be transformed to a device-independent color space which is consistent with the color space of the video imaging device used to acquire the video. Upon convergence, the last vector of estimated skin parameters Pₑ (208) is determined to be the intermediate vector of estimated skin parameters P_{med}. The intermediate vector and the iteration history are stored to storage device 210 and further processing stops.

If convergence has not yet occurred then Optimizer-A 207 generates a next vector of estimated skin parameters 208 to be provided to the biophysics model 209 on a next iteration. Optimizer-A further varies any of the values of any of the parameters in the initial vector of skin parameters Pᵢₙᵢₜ such that a next vector of estimated skin parameters Pₑ generated thereby is different than any of the previously generated vectors of estimated skin parameters. On each iteration, the biophysics model receives (as input) a vector of estimated skin parameters 208 and generates (as output) a vector of estimated skin parameters 205. The process repeats until convergence. Upon convergence, the last vector of estimated color measurements 205 will closely match the vector of time-averaged in-vivo color measurements 203. An exact match between the two vectors will likely not be achieved due to a variety of factors including noise in the system. The intermediate vector of skin parameters P_{med} and the iteration history are stored to storage device 210. The intermediate vector is used in FIG. 3 on a first iteration.

### Functional Block Diagram of FIG. 3

Reference is now being made to the functional block diagram of FIG. 3 which implements a system for determining a final vector of estimated skin parameters for each image frame of the video. The embodiment of FIG. 3 repeats for each image frame of the video. The intermediate vector of estimated skin parameters obtained by the block diagram of FIG. 2 is used on a first iteration to obtain a first vector of estimated color measurements 305.

In FIG. 3, in-vivo color measurements 303 from the current image frame being processed are compared 304 against the vector of estimated color measurements 305 to determine if convergence has occurred. Convergence occurs if the error 306 is at or near zero. As discussed with respect to the embodiment of FIG. 2, convergence can be determined using, for example, a norm of an error vector, a sum of quadratic errors, a natural log of a sum of quadratic errors, a maximum square error, a mean square error, and a Kullback-Leibler divergence metric. If the color spaces of the two vectors being compared are different then the estimated color measurements can be transformed to a device-independent color space which is consistent with the color space of the video imaging device used to acquire the video. If convergence has occurred then the last vector of estimated skin parameters 308 is determined to be the final vector of estimated skin parameters P_{final} for the current image frame. The final vector of estimated skin parameters P_{final} and the iteration history are saved to storage device 310. Processing then continues for the next image frame in the video until a final vector P_{final} has been obtained for each image frame and stored.

If convergence has not yet occurred then Optimizer-B 307 generates a next vector of estimated skin parameters Pₑ 308 in a similar manner as Optimizer-A 207 with the difference being that Optimizer-B varies any of the time-varying skin parameters in the intermediate vector while holding the time-invariant parameters fixed. The next vector of estimated skin parameters 308 is then provided to the biophysics model 309 which, in turn, generates a next vector of estimated skin parameters 305. The iterative process repeats for the current image frame until convergence has occurred. Upon convergence, the last vector of estimated skin parameters Pₑ 308 is determined to be the final vector of estimated skin parameters P_{final} for this image frame. The final vector of estimated skin parameters and the iteration history are stored to storage device 310.

Many of the functional blocks of FIGS. 2 and 3 perform the same or nearly the same function(s). The embodiments of FIGS. 2 and 3 are shown as separate diagrams for explanatory purposes. Thus, a block diagram which is a combination of FIGS. 2 and 3 can be readily obtained. As such, the diagrams of FIGS. 2 and 3 should not be viewed as limiting the scope of the appended claims strictly to these two embodiments.

It should be appreciated that the steps of “determining”, “averaging”, “receiving”, “processing”, “generating”, “comparing”, and the like, as used herein, include the application of any of a variety techniques as well as mathematical operations according to any specific context or for any specific purpose. It should be appreciated that such steps may be facilitated or otherwise effectuated by a microprocessor executing machine readable program instructions such that an intended functionality can be effectively performed.

### Example Flow Diagram

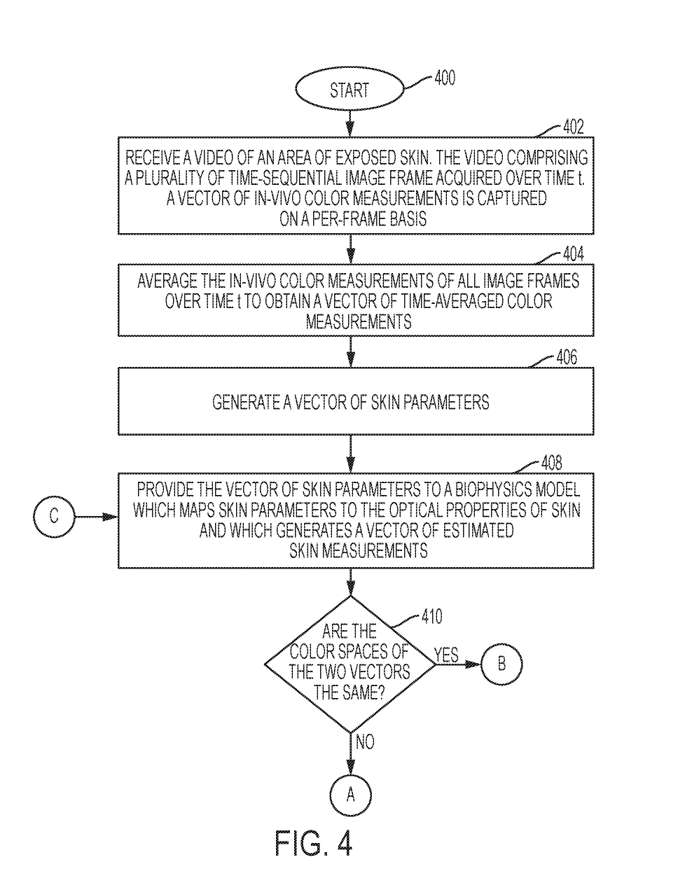

Reference is now being made to the flow diagram of FIG. 4 which illustrates one example embodiment of the present method for estimating a final vector of skin parameters for each image from of a video of a skin surface of a subject. Flow processing begins at step 400 and immediately proceeds to step 402.

At step 402, receive a video of an area of exposed skin of a subject. The video comprises a plurality of time-sequential image frames acquired over time t. A vector of in-vivo color measurements of the skin are acquired on a per-frame basis. FIG. 1 shows an example video imaging device capturing in-vivo color measurements of a skin surface of a subject's hand.

At step 404, average the in-vivo color measurements of all image frames over time t to obtain a vector of time-averaged in-vivo color measurements.

At step 406, generate a vector of estimated skin parameters.

At step 408, provide the vector of estimated skin parameters to a biophysics model which maps skin parameters to the optical properties of skin and which generates a vector of estimated color measurements.

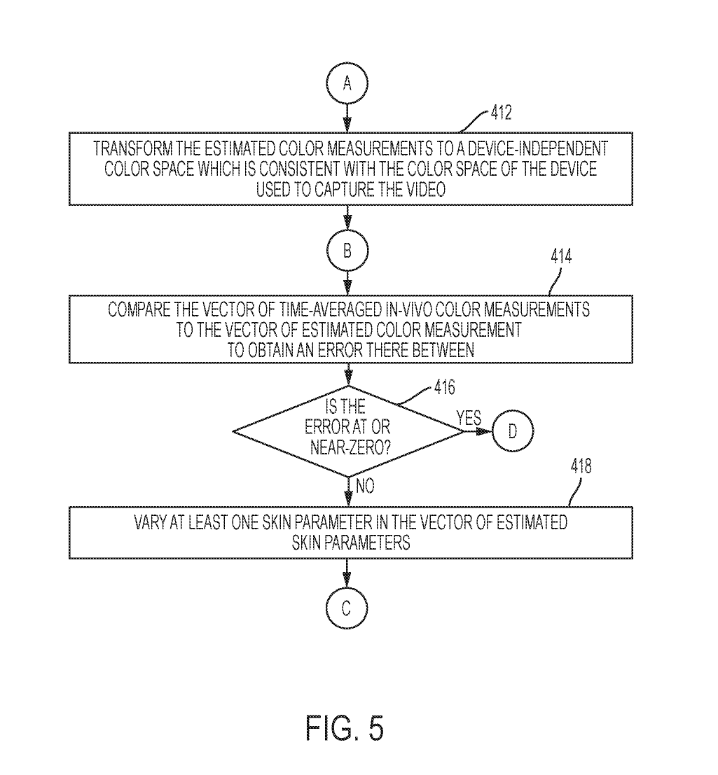

At 410, a determination is made whether the color spaces of the estimated color measurements and the in-vivo color measurements are the same. If not then processing continues with respect to node A of FIG. 5. Otherwise, processing continues with respect to node B of FIG. 5.

Reference is now being made to the flow diagram of FIG. 5 which is a continuation of the flow diagram of FIG. 4 with flow processing continuing with respect to node A or B.

At step 412, transform the estimated color measurements to a device-independent color space which is consistent with the color space of the video imaging device used to acquire the video.

At step 414, compare the vector of time-averaged in-vivo color measurements to the vector of estimated color measurements to obtain an error there between.

At step 416, a determination is made whether the error is at or near-zero. If the error is at or near zero then processing continues with respect to node D.

At step 418, vary at least one skin parameter in the vector of estimated skin parameters. Thereafter, processing continues with respect to node C wherein, at step 408, the vector of estimated skin parameters is again provided to the biophysics model to obtain a next vector of estimated color measurements. Processing repeats in a similar manner until the error between the vector of estimated color measurements and the time-averaged in-vivo color measurements is at or near zero.

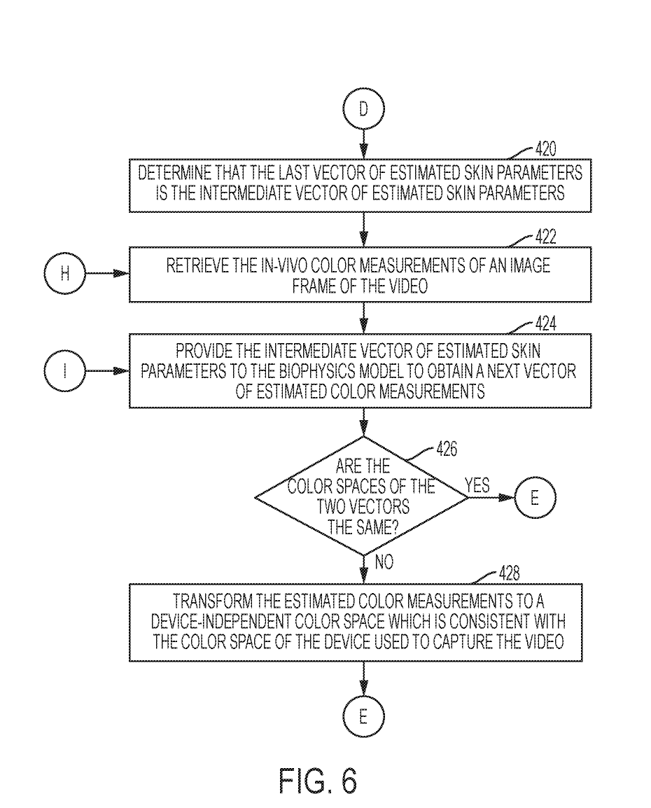

Reference is now being made to the flow diagram of FIG. 6 which is a continuation of the flow diagram of FIG. 5 with flow processing continuing with respect to node D.

At step 420, determine that a last vector of estimated skin parameters (from the last iteration) is the intermediate vector of estimated skin parameters. The following steps repeat for all image frames in the video.

At step 422, retrieve the in-vivo color measurements of an image frame of the video. It should be appreciated that, on a first iteration, the in-vivo color measurements associated with first image frame are retrieved. On a next iteration, the in-vivo color measurements associated with a next image frame are retrieved, and so on.

At step 424, provide the intermediate vector of estimated skin parameters (from step 420) to the biophysics model to obtain a next vector of estimated color measurements.

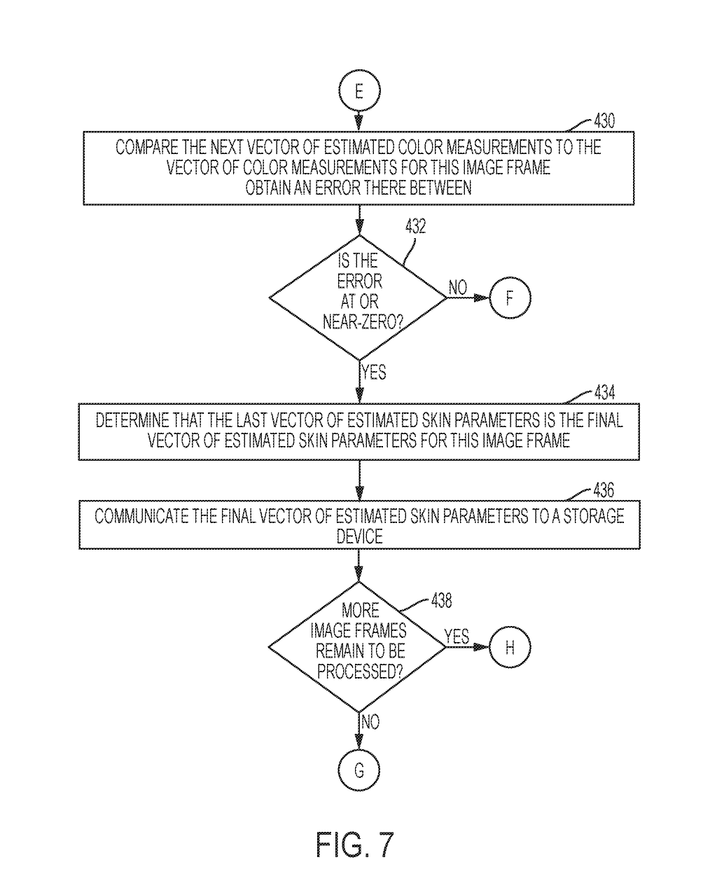

At step 426, a determination is made whether the color spaces of the estimated color measurements and the in-vivo color measurements are the same. If the two color spaces are the same then processing continues with respect to node E of FIG. 7.

At step 428, transform the estimated color measurements to a device-independent color space which is consistent with the color space of the video imaging device used to acquire the video.

Reference is now being made to the flow diagram of FIG. 7 which is a continuation of the flow diagram of FIG. 6 with processing continuing with respect to node E.

At step 430, compare the next vector of estimated color measurements to the vector of in-vivo color measurements (for this frame) to obtain an error there between.

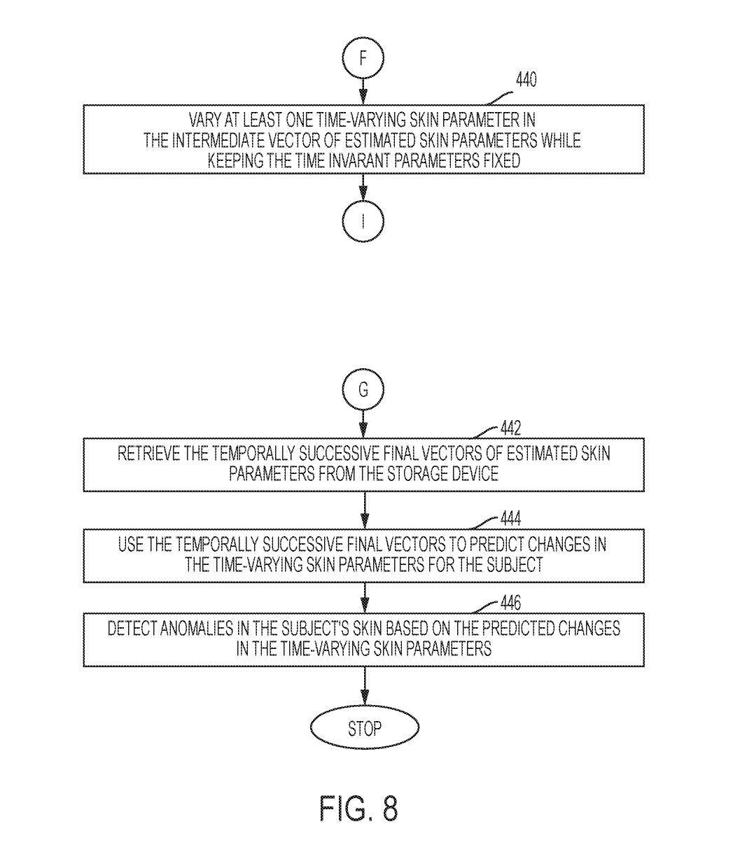

At step 432, a determination is made whether the error is at or near-zero. If the error is not at or near zero then processing continues with respect to node F of FIG. 8. Otherwise, processing continues with respect to step 434.

At step 434, determine that estimated skin parameters (from the previous iteration) is the final vector of estimated skin parameters for this image frame.

At step 436, communicate the final vector of estimated skin parameters to a storage device. An example storage device is shown and discussed with respect the block diagrams of FIGS. 2 and 3.

At step 438, a determination is made whether more image frames remain to be processed. If no more image frames remain to be processed then processing continues with respect to node G of FIG. 8. Otherwise, processing repeats with respect to node H of FIG. 6 wherein, at step 422, retrieve the in-vivo color measurements of a next image frame. Processing repeats for this next image frame until no more image frames remain to be processed.

Reference is now being made to the flow diagram of FIG. 8 which is a continuation of the flow diagram of FIG. 7 with processing continuing with respect to nodes F or G.

At step 440, vary at least one time-varying skin parameter in the intermediate vector of estimated skin parameters while keeping the time invariant parameters fixed. Processing continues with respect to node I wherein, at step 424, provide the intermediate vector of estimated skin parameters to the biophysics model to obtain a next vector of estimated color measurements. Processing repeats in a similar manner until, at step 432, the error is at or near zero and a final vector of estimated skin parameters has been obtained for this image frame and communicated to a storage device.

At step 442, retrieve the temporally successive final vectors of estimated skin parameters (obtained on a per-frame basis) from the storage device.

At step 444, use the temporally successive final vectors to predict changes in the time-varying skin parameters for the subject.

At step 446, detect anomalies in the subject's skin based on the predicted changes in the time-varying skin parameters. Thereafter, in this embodiment, further processing stops.

Anomalies present in the skin of the subject can be detected by monitoring changes in the temporally sequential final vectors of estimated skin parameters over time. This can be effectuated using, for example, process control monitoring where Principal Component Analysis (PCA) is used to map estimated skin parameters to an one-dimensional value. The mapping can be achieved using the T² Hotelling or the Squared Prediction Error. Confidence limits can be computed for either T² Hotelling or Squared Prediction Error using only estimated skin parameters of a subject who is healthy. The mapping of estimated skin parameters to T² Hotelling or Squared Prediction Error can be performed in real-time and, if the value is greater than the confidence limit, then an anomaly is detected. The Q-contribution plot can be used to identify which estimated skin parameter is creating the anomaly.

A diagnosis of the skin of a subject can be made using, for example, a classification algorithm that indicates whether a subject is healthy or not. Data sets of skin parameters that correspond to healthy subjects or subjects who have developed different skin diseases can be utilized to train the classifier off-line. Each data set corresponds to a particular case. If there are one healthy subject and three subjects with different diseases then there are four data sets in the classifier. The trained classifier is then used for processing a sequence of real-time estimated skin parameters to diagnose the subject's skin ailment, if any. Alternatively, a time-series analysis combined with a Laplace transform is used to diagnose the skin of a subject. One skilled in this art would appreciate that this generates a transfer function that contains zeroes and poles. Data sets of skin parameters that correspond to subjects who are healthy or who have developed different skin diseases are used to derive the location of the zeroes and poles for each case. The zeroes and poles locations obtained in real-time can be compared to the off-line zeroes and poles locations and a diagnostic can be made by selecting a minimum Euclidean distance out of all the distances between the real-time and the off-line locations.

It should be appreciated that the flow diagrams depicted herein are illustrative. One or more of the operations in the flow diagrams may be performed in a differing order. Other operations may be added, modified, enhanced, or consolidated. Variations thereof are intended to fall within the scope of the appended claims.

### Example Networked System

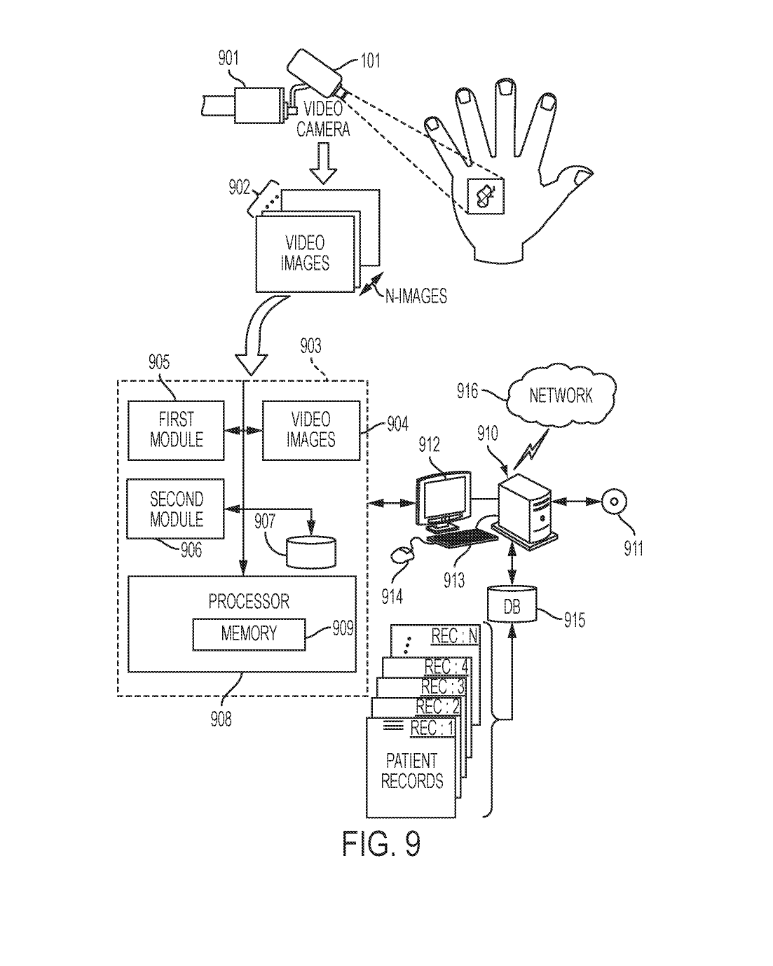

Reference is now being made to FIG. 9 which shows a functional block diagram of one embodiment of an example video processing system for performing various aspects of the present method as described with respect to the block diagrams of FIGS. 2 and 3 and the flow diagrams of FIGS. 4-8.

In FIG. 9, video camera 101, shown rotatably mounted on a robotic arm 901 which moves the video camera so that the medical practitioner can focus the camera on an area of exposed skin containing, for example, a skin lesion. Video images (collectively at 902) are communicated to system 903. Video Processor 904 processes the image frames of the video to obtain in-vivo color measurements on a per-frame basis. Video Processor 904 also uses any of a variety of image processing methods to facilitate a location and identification of the area of interest on the hand. First Module 905 performs the functionality shown and described with respect to FIG. 2. Second Module 906 performs the functionality shown and described with respect to FIG. 3. In other embodiments, the First and Second Modules are the same. The intermediate and final vectors of estimated skin parameters are stored/retrieved to storage device 907. Processor 908 retrieves machine readable program instructions from Memory 909 to facilitate the functionality of any of the modules of system 903. It should be appreciated that some or all of the functionality of system 903 can be incorporated within the video camera 101. System 903 is shown in communication with a workstation 910.

Workstation 910 has a computer case which houses various components such as, for instance, a motherboard with a processor and memory, a communications link such as a network card, a video card, an internal hard drive capable of reading/writing to machine readable media 911 such as a floppy disk, optical disk, CD-ROM, DVD, magnetic tape, and the like, and other software and hardware needed to perform the functionality of a computing system. The workstation further includes a display device 912, such as a CRT, LCD, or touchscreen device, for displaying information, video, measurement data, computational values, patient medical information, results, including distances, locations, and the like. A user can view that information and make a selection from menu options displayed thereon. Keyboard 913 and mouse 914 effectuate a user input or selection.

Workstation 910 has an operating system and other specialized software configured to display alphanumeric values, menus, scroll bars, dials, slideable bars, pull-down options, selectable buttons, and the like, for entering, selecting, modifying, and accepting information needed for processing video images and for enabling a medical practitioner to perform a medical diagnosis. Software to configure a user interface or any portion thereof to display/enter/accept data is generally customizable. A user or technician of the workstation may use the user interface to identify regions of interest, set parameters, use a rubber-band box to select portions of image frames, and/or regions of images for processing. These selections may be stored and retrieved from storage device 907 and/or computer readable media 911. Default settings and initial parameters can be retrieved from storage device 915, as needed.

Workstation 910 implements database 915 wherein records are stored, manipulated, and retrieved in response to a query. Such records, in various embodiments, take the form of medical histories stored in association with information identifying the patient, the video, color measurements obtained, and the like. Information regarding mathematical representations and data values used to perform any aspects of the methods disclosed herein can also be stored along with these records. Although the database is shown as an external device, the database may be internal to the workstation mounted, for example, on a hard disk therein.

Although shown as a desktop computer, the workstation can be a laptop, a mainframe, a client/server, or a special purpose computer such as an ASIC, circuit board, dedicated processor, or the like. The embodiment of the workstation is illustrative and may include other functionality known in the arts. Any of the components of the networked workstation may be placed in communication with Control System 903. Any of the modules and processing units of the system 903 can be placed in communication with storage device 915 or computer readable media 911 and may store/retrieve therefrom data, variables, records, parameters, functions, and/or machine readable/executable program instructions, as required to perform their intended functions.

Each of the modules of the system 903 may be placed in communication with one or more remote devices over network 916. It should be appreciated that some or all of the functionality performed by any of the modules or processing units of system 903 can be performed, in whole or in part, by workstation 910 or by a workstation placed in communication with the system 903 over network 916. The embodiment shown is illustrative and should not be viewed as limiting the scope of the appended claims strictly to the configuration shown. In other embodiments, the generated results are provided to a server over the network and communicated to multiple user/operators in various diverse locations.

Various modules may designate one or more components which may, in turn, comprise software and/or hardware designed to perform the intended function. A plurality of modules may collectively perform a single function. Each module may have a specialized processor capable of executing machine readable program instructions. A module may comprise a single piece of hardware such as an ASIC, electronic circuit, or special purpose processor. A plurality of modules may be executed by either a single special purpose computer system or a plurality of special purpose computer systems in parallel. Connections between modules include both physical and logical connections. Modules may further include one or more software/hardware modules which may further comprise an operating system, drivers, controllers, MIMO's, inverters, optimizers, some or all of which may be connected via a network. It is also contemplated that one or more aspects of the present method may be implemented on a dedicated computer system and may also be practiced in distributed computing environments where tasks are performed by remote devices that are linked via network 916.

The teachings hereof can be implemented in hardware or software using any known or later developed systems, structures, devices, and/or software by those skilled in the applicable art without undue experimentation from the functional description provided herein with a general knowledge of the relevant arts. Moreover, the teachings hereof may be partially or fully implemented in software using object or object-oriented software development environments that provide portable source code that can be used on a variety of computer, workstation, server, network, or other hardware platforms. One or more of the capabilities hereof can be emulated in a virtual environment as provided by an operating system, specialized programs or leverage off-the-shelf computer graphics software such as that in Windows, Java, or from a server or hardware accelerator or other image processing devices.

One or more aspects of the methods described herein are intended to be incorporated in an article of manufacture, including one or more computer program products, having computer usable or machine readable media. The article of manufacture may be included on at least one storage device readable by a machine architecture embodying executable program instructions capable of performing the methodology described herein. The article of manufacture may be shipped, sold, leased, or otherwise provided separately either alone or as part of a product suite or a service.

It will be appreciated that the above-disclosed and other features and functions, or alternatives thereof, may be desirably combined into many other different systems or applications. Various presently unforeseen or unanticipated alternatives, modifications, variations, or improvements therein may become apparent and/or subsequently made by those skilled in the art which are also intended to be encompassed by the following claims. The teachings of any printed publications including patents and patent applications are each separately hereby incorporated by reference in their entirety.

1. A method for estimating a vector of skin parameters from in-vivo color measurements obtained from a video of a skin surface, the method comprising:
receiving a video of an area of exposed skin of a subject, the video comprising a plurality of time-sequential image frames acquired over time t, a vector of in-vivo color measurements of the skin being obtained on a per-frame basis from at least one imaging channel of a video imaging device used to capture the video;
determining, from the video, an intermediate vector of estimated skin parameters based on an initial vector of estimated skin parameters and the in-vivo color measurements of all image frames averaged over time t;
for each image frame in the video, determining a final vector of estimated skin parameters based on the intermediate vector of estimated skin parameters and the in-vivo color measurements of the current frame; and
using the temporally successive final vectors to predict changes in time-varying skin parameters for the subject.

2. A method of claim 1, wherein determining the intermediate vector of estimated skin parameters comprises:
averaging, over time t, the in-vivo color measurements of all image frames to obtain a vector of time-averaged in-vivo color measurements;
generating an initial vector of estimated skin parameters;
(A) providing the vector of estimated skin parameters to a biophysics model which maps skin parameters to the optical properties of skin and which generates a vector of estimated color measurements as output;
(B) comparing the vector of time-averaged in-vivo color measurements to the vector of estimated color measurements to obtain an error there between;
(C) in response to the error being near-zero, determining that a last vector of estimated skin parameters is the intermediate vector of estimated skin parameters, otherwise varying at least one parameter in the vector of estimated skin parameters, and repeating (A)-(C).

3. The method of claim 1, wherein the initial vector of estimated skin parameters contains at least one skin parameter that varies over time t and at least one skin parameter that does not vary over time t.

4. The method of claim 3, wherein the time-invariant skin parameters are at least one of: epidermal thickness and melanin concentration, and wherein the time-varying skin parameters are at least one of: dermal blood volume fraction, skin oxygen saturation, and a light scattering parameter.

5. The method of claim 2, wherein, in response to the color space of the vector of estimated color measurements generated by the biophysics model being different than the color space of the in-vivo color measurements, transforming the estimated color measurements to a device-independent color space which is consistent with the color space of the video imaging device used to acquire the video.

6. The method of claim 2, wherein varying at least one parameter in the vector of estimated skin parameters is performed using any of: a Nelder-Mead Algorithm, a Sequential Quadratic Programming, a Genetic Algorithm, a Constrained Levenberg-Marquard Algorithm, and a Simultaneous Perturbation Stochastic Approximation.

7. A method of claim 1, wherein determining the final vector of estimated skin parameters comprises:
(A) providing the intermediate vector of estimated skin parameters to a biophysics model which maps skin parameters to the optical properties of skin and which generates a vector of estimated color measurements as output;
(B) comparing the vector of estimated color measurements to the vector of in-vivo color measurements for this frame to obtain an error there between;
(C) in response to the error being near-zero, determining that a last vector of estimated skin parameters is the final vector of estimated skin parameters for this frame, otherwise varying at least one time-varying skin parameter in the intermediate vector of estimated skin parameters while keeping the time invariant parameters fixed, and repeating (A)-(C).

8. The method of claim 7, wherein, in response to the color space of the estimated color measurements generated by the biophysics model being different than the color space of the in-vivo color measurements, transforming the estimated color measurements to a device-independent color space which is consistent with the color space of the video imaging device used to acquire the video.

9. The method of claim 7, wherein varying at least one time-varying skin parameter in the intermediate vector of estimated skin parameters uses any of: a Nelder-Mead Algorithm, a Sequential Quadratic Programming, a Genetic Algorithm, a Constrained Levenberg-Marquard Algorithm, and a Simultaneous Perturbation Stochastic Approximation.

10. The method of claim 1, wherein the in-vivo color measurements are from any of: a measurement obtained for a single pixel, measurements obtained for a group of pixels, and measurements obtained for different groups of pixels.

11. The method of claim 1, further comprising using the predicted changes in the time-varying skin parameters to detect anomalies present in the subject's skin.

12. A system for estimating a vector of skin parameters from in-vivo color measurements obtained from a video of a skin surface, the system comprising:
a storage device; and
a processor in communication with said storage device, said processor executing machine readable instructions for performing:
receiving a video of an area of exposed skin of a subject, the video comprising a plurality of time-sequential image frames acquired over time t, a vector of in-vivo color measurements of the skin being obtained on a per-frame basis from at least one imaging channel of a video imaging device used to capture the video;
determining, from the video, an intermediate vector of estimated skin parameters based on an initial vector of estimated skin parameters and the in-vivo color measurements of all image frames averaged over time t;
for each image frame in the video, determining a final vector of estimated skin parameters based on the intermediate vector of estimated skin parameters and the in-vivo color measurements of the current frame; and
using the temporally successive final vectors to predict changes in time-varying skin parameters for the subject.

13. A system of claim 12, wherein determining the intermediate vector of estimated skin parameters comprises:
averaging, over time t, the in-vivo color measurements of all image frames to obtain a vector of time-averaged in-vivo color measurements;
generating an initial vector of estimated skin parameters;
(A) providing the vector of estimated skin parameters to a biophysics model which maps skin parameters to the optical properties of skin and which generates a vector of estimated color measurements as output;
(B) comparing the vector of time-averaged in-vivo color measurements to the vector of estimated color measurements to obtain an error there between;
(C) in response to the error being near-zero, determining that a last vector of estimated skin parameters is the intermediate vector of estimated skin parameters, otherwise varying at least one parameter in the initial vector of estimated skin parameters, and repeating (A)-(C).

14. The system of claim 12, wherein the initial vector of estimated skin parameters contains at least one skin parameter that varies over time t and at least one skin parameter that does not vary over time t.

15. The system of claim 14, wherein the time-invariant skin parameters are at least one of: epidermal thickness and melanin concentration, and wherein the time-varying skin parameters are at least one of: dermal blood volume fraction, skin oxygen saturation, and a light scattering parameter.

16. The system of claim 13, wherein, in response to the color space of the vector of estimated color measurements generated by the biophysics model being different than the color space of the in-vivo color measurements, transforming the estimated color measurements to a device-independent color space which is consistent with the color space of the video imaging device used to acquire the video.

17. The system of claim 13, wherein varying at least one parameter in the vector of estimated skin parameters is performed using any of: a Nelder-Mead Algorithm, a Sequential Quadratic Programming, a Genetic Algorithm, a Constrained Levenberg-Marquard Algorithm, and a Simultaneous Perturbation Stochastic Approximation.

18. A system of claim 12, wherein determining the final vector of estimated skin parameters comprises:
(A) providing the intermediate vector of estimated skin parameters to a biophysics model which maps skin parameters to the optical properties of skin and which generates a vector of estimated color measurements as output;
(B) comparing the vector of estimated color measurements to the vector of in-vivo color measurements for this frame to obtain an error there between;
(C) in response to the error being near-zero, determining that a last vector of estimated skin parameters is the final vector of estimated skin parameters for this frame, otherwise varying at least one time-varying skin parameter in the intermediate vector of estimated skin parameters while keeping the time invariant parameters fixed, and repeating (A)-(C).

19. The system of claim 18, wherein, in response to the color space of the estimated color measurements generated by the biophysics model being different than the color space of the in-vivo color measurements, transforming the estimated color measurements to a device-independent color space which is consistent with the color space of the video imaging device used to acquire the video.

20. The system of claim 18, wherein varying at least one time-varying skin parameter in the intermediate vector of estimated skin parameters uses any of: a Nelder-Mead Algorithm, a Sequential Quadratic Programming, a Genetic Algorithm, a Constrained Levenberg-Marquard Algorithm, and a Simultaneous Perturbation Stochastic Approximation.
